(19) United States
(12) Patent Application Publication
(10) Pub. No.: US 2010/0047766 A1
(43) Pub. Date: Feb. 25, 2010

(54) ANALYTE MANIPULATION AND DETECTION

(75) Inventors: Denise Barrault, Midlothian (GB); Stuart Polwart, Midlothian (GB); David Thomson, Midlothian (GB)

Correspondence Address: Joseph R. Baker, APC;Gavrilovich, Dodd & Lindsey LLP, 4660 La Jolla Village Drive, Suite 750, San Diego, CA, 92122, US

(73) Assignee: ITI SCOTLAND LIMITED, Glasgow, GB (GB)

(21) Appl. No.: 12/524,779

(22) PCT Filed: Jan. 29, 2008
(86) PCT No.: PCT/EP2008/051040

§ 371 Date: Oct. 2, 2009

(30) Foreign Application Priority Data
Jan. 29, 2007 (GB) 0701641.3

(51) Int. Cl. C12Q 1/70; G01N 33/53; C12M 1/34; C40B 40/00; C12Q 1/68

(52) U.S. Cl. 435/5; 435/7.2; 435/287.1; 435/287.2; 506/13; 435/6

(57) ABSTRACT

Provided is a method for separating two or more analytes in a fluid, which method comprises:
(a) binding each different analyte to a different particle in a binding zone, to produce two or more bound analytes;
(b) allowing the bound analytes to move through a separating conduit to two or more separate functional conduits;
   wherein each different particle has, or can be controlled to have, a different buoyancy in the fluid as compared with the other particles; and wherein the separating conduit is in fluid communication with the two or more functional conduits, each functional conduit being situated at a different height from the other functional conduits; and each functional conduit containing a fluid having a different fluid density from the fluids in the other functional conduits such that each different particle when attached to an analyte has neutral buoyancy in at least one of the functional conduits, thereby allowing separation of the bound analytes by means of the neutral buoyancies of the different particles in the different functional conduits.

The present invention concerns methods for manipulating and detecting analytes. The method relates in particular to methods for separating different analytes from the same sample. The invention is particularly advantageous, since its separation aspects allow a plurality of different analytes in a single sample to be detected, or separately manipulated.

It has been known to employ buoyant particles, and other types of particles (such as magnetic particles and high density particles) in methods of analysis, in particular in biological assays. In addition to this, methods using buoyant beads to remove waste from the surface of large volumes of water, such as swimming pools are also well-known and have been developed by MST (MicroScience Technologies). Typically, hollow particles that are buoyant and are capable of attaching to bacterial contaminants in the water via an antibody linked to the surface of the particle are mixed with the water and upon rising to the surface, the bacteria and particle mixture is ‘skimmed’ from the surface to detect the pool contaminants. This has been carried out for detection of cryptosporidium in swimming pools.

Methods for detecting analytes using magnetic particles, in particular magnetic beads, have been around for some time. For example, common assay methods use magnetic beads which are added to the sample to be assayed. The beads carry a ligand on their surface which enables them to bind specifically to a target analyte. A magnetic field is then applied, enabling the beads and the bound material to be separated from the rest of the sample. In many cases the analyte is then measured by detection of a fluorescence-based emission, and can be used in conjunction with flow cytometric analysis. Such methods have been used for in vitro diagnostics against desired targets such as cells, nucleic acids, proteins and other types of biomolecule.

However, these types of existing methods require several sample preparation steps. In particular, this invention allows for testing and separation of several analytes within one reaction volume, or platform in the case of a microfluidic system. Further, previous techniques have often required several reactions in parallel to achieve multi-analyte reactions, resulting in lack of consistency and irreproducibility of results. Also relevant to a microfluidic lab-on-a-chip system, or integrated sample analysis device, this capability allows analytes to be captured as a whole from the entire sample volume. The captured analytes can then be separated towards different processing modules within the sample. The advantage here is that the capture step is carried out on the full volume of the collected sample, rather than an aliquot of the sample, which is the case in most analytical systems where the sample is split. This means that at equal sensitivity, lower volumes of sample are needed, providing substantial benefits to the patient, who accordingly need not provide such a large specimen.

It is an aim of the present invention to solve the problems associated with known techniques, including those described above. It is a further aim of the invention to develop improved methods for processing analytes (such as separating analytes) and detecting analytes.

Accordingly, the present invention provides a method for separating two or more analytes in a fluid, which method comprises:
(a) binding each different analyte to a different particle in a binding zone, to produce two or more bound analytes;
(b) allowing the bound analytes to move through a separating conduit to two or more separate functional conduits;
   wherein each different particle has, or can be controlled to have, a different buoyancy in the fluid as compared with the other particles; and wherein the separating conduit is in fluid communication with the two or more functional conduits, each functional conduit being situated at a different height from the other functional conduits; and each functional conduit containing a fluid having a different fluid density from the fluids in the other functional conduits such that each different particle when attached to an analyte has neutral buoyancy in at least one of the functional conduits, thereby allowing separation of the bound analytes by means of the neutral buoyancies of the different particles in the different functional conduits.

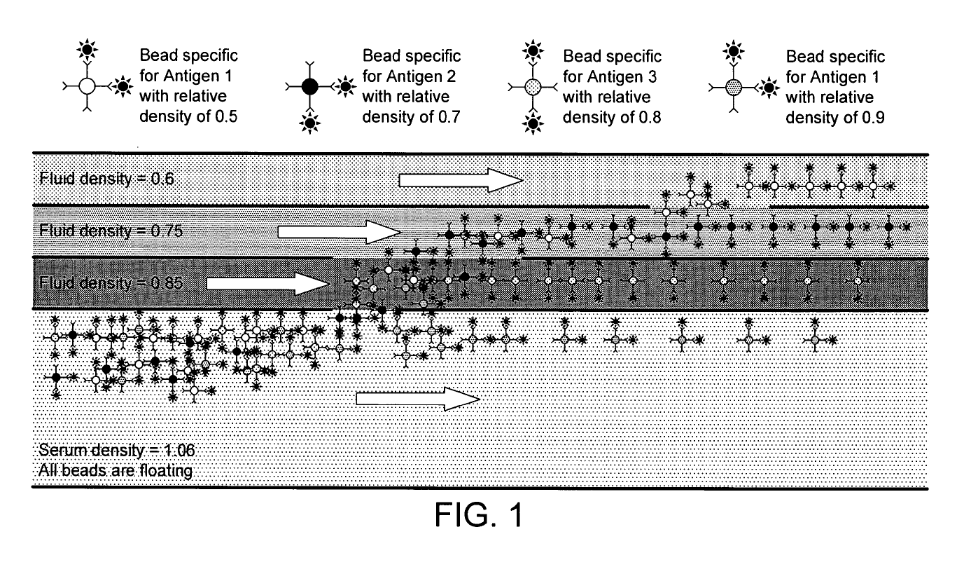

An example of the invention in operation is depicted in FIG. 1. The Figure shows the separating conduit, which in this preferred embodiment of the invention is comprised of four functional conduits. The functional conduits are arranged one above the other, and comprise four different fluids, each fluid having a different density. The lowest density fluid is situated at the highest position, whilst the highest density fluid is situated in the lowest position, in order that the separation is most effective. There are four different analytes to be separated in this example, and each different analyte is attached to a different particle, in this case hollow beads, by means of an antibody specific for an antigen on the analyte. The antibodies are attached to different beads before commencing the separation method. Each different bead has a neutral buoyancy in one of the fluids in the functional conduits, and thus the analytes will rise through the connecting regions between the functional conduits, until they reach a level at which they are neutrally buoyant, at which point they will no longer rise or fall and will be channelled into separate regions of the functional conduit. As an alternative, the beads may sink through lower and lower functional conduits until they reach a level at which they are neutrally buoyant. It is further possible that both embodiments may be combined and some beads may sink and some may rise. From these regions the separated analytes may be further processed, or detected as desired. Although the functional conduits are in fluid connection with each other, they are deemed to be separate from each other in the present context, because they possess regions beyond the fluid connection that are capable of keeping the analytes separate.

In the context of the present invention, neutrally buoyant is a situation where 95% or more of the beads of a certain type (combination of density, recognition molecule and bound analyte) remain (do not leave whilst flowing) in a particular channel (functional conduit) of the platform.

Preferably, the separating conduit is connected to the binding zone by a connecting conduit along which the bound analytes are allowed to flow, until they reach the separating conduit. Typically, the connecting conduit forms the lowest functional conduit (or highest functional conduit in the ‘sinking’ embodiment) and at least one particle is thus neutrally buoyant in the fluid that is used as a medium for binding and for transporting the bound analytes to the separating conduit (as shown in FIG. 1). However, in other embodiments of the invention, the connecting conduit may not be employed as a functional conduit for separation, and in such embodiments all of the analytes rise (or fall) out of the connecting conduit into the functional conduits above (or below), for separating. It will be apparent from this example that the separating conduit may be comprised of the functional conduits (either as separate elements joined together, or as a single integral element).

It will be understood that it is a particularly preferred feature of the present invention that successively higher functional conduits contain successively lower density fluids, that is to say that the fluid density in each successively higher conduit becomes progressively lower (or vice versa in the ‘sinking’ embodiment). However, this feature is not essential. For example in the case of two analytes and three conduits (a high density conduit situated between two lower density conduits) separation will still be effective if the lowest conduit has a higher density fluid than the highest conduit, provided that one analyte has neutral buoyancy in the lowest conduit, and will thus remain there, whilst the second analyte has neutral buoyancy in the highest conduit, and will rise through the lowest and central conduit into the highest conduit. Nevertheless, this is a less efficient configuration and it is much preferred in a normal situation that the fluid density in each successively higher conduit becomes progressively lower in order that the most efficient separation can be achieved.

The functional conduits are not especially limited, provided that they are each capable of containing their fluid, without it mixing with fluid from a connecting functional conduit at the connecting locations. Generally, the conduits are of dimensions typical in microfluidic devices, since these dimensions are particularly suitable for reducing turbulence in the flowing fluids, and thus minimising any tendency to mixing at the locations where there is fluid connection between the functional conduits. Typical conduit dimensions are around 50-1000 μm. In fact, on this scale mixing at the fluid connection points is minimal, and certainly insufficient to have an adverse effect on the separation process (see Kamholz, A. E., Weigl, B. H., Finlayson, B. A. & Yager, P. “Quantitative analysis of molecular interaction in a microfluidic channel: The T-sensor.” Analytical Chemistry 71, 5340-5347, 1999).

As mentioned above, the functional conduits are in fluid connection with each other in order to achieve separation. These fluid connections are not especially limited provided that they are of sufficient size to allow passage of the particles and analytes from one functional conduit to the next, but not so large that mixing of fluids of different density occurs. Preferably the connections are slightly longer than the width of the channels, and form an open window on the whole of the superior surface (shadow cast vertically by the shape of the channel transporting the fluid) of the channel where the beads are in motion.

In the present method, the fluid may be any suitable fluid. Preferably, the fluid is an aqueous fluid.

In a preferred embodiment of the method, after step (a), the method further comprises transporting the bound analyte from one or more of the separate functional conduits where separation has taken place, to a concentrating zone beneath one or more detection elements in the fluid.

Each particle employed in the method is attached to a recognition agent. The number of different recognition agents will depend on the number of analytes in the sample that are under investigation. Usually each different particle type is attached to a different recognition agent, to ensure that one type of analyte attaches to one type of particle, and the other types of analyte each attach to other different types of particle. In alternative embodiments, each particle may be attached to more than one recognition agent, so that the analytes are separated into groups rather than individual types. The recognition agents attached to a single particle may be the same (for example if it is desirable to increase the binding potential of the particle to the analyte, or to attach more than one analyte species to a single particle) or may be different (e.g. if it is desirable to attach any of the analytes under investigation to any of the particles). In some of the latter embodiments, all of the different types of recognition agent in the system may be attached to a single particle so that any or all of the possible analytes may bind to a single particle.

In the present invention, the particles are not especially limited, provided that their function is not impaired. Preferably, the particles are selected from:
(a) particles that are buoyant in the fluid;
(b) magnetic particles whose buoyancy can be controlled by the application of a magnetic field;
(c) particles that are more dense than the fluid; and/or
(d) particles that are neutrally buoyant in the fluid.

The latter particles may be used for the lowest level functional conduit, particularly if the lowest level conduit is formed from the connecting conduit such that the particles are not required to rise to collect in this conduit. Magnetic particles may also be employed, and are especially useful if variance in the buoyancy is desired, for example to fine tune the separation efficiency. However, typically sets of buoyant particles having differing buoyancies is desired, such as glass beads having a controlled range of buoyancies.

The method of the present invention is advantageous because it allows a more rapid separation (and detection) of analytes in a sample by reducing the number of processing steps conducted on the sample. Further, it reduces the amount of laboratory equipment required, making the method easier, and less costly, to perform. The invention is particularly advantageous, since its separation aspects allow a plurality of different analytes in a single sample to be detected, or separately manipulated. It also allows the whole of the sample to be accessed by each bead type, rather than having to split the sample into different aliquots which is the case in traditional testing methods. Once the analytes of interest have been captured by the recognition elements on the bead, and separated by the fluids present in the different conduits, they can easily be processed separately, potentially using very different methods. These properties are of particular interest on analytical instruments which carry out multi-analyte testing or on integrated lab-on-a-chip systems.

The present invention will be described by way of example only, with reference to the following Figure:

FIG. 1 shows, as a schematic, an example of the layout of a separating apparatus for detection of four analytes in one embodiment of the present invention, using the connecting conduit containing serum (density 1.06) as the lowest functional conduit, and three further functional conduits above this with fluids of density 0.85, 0.75 and 0.6

The invention will now be described in more detail.

The methods of the present invention may be employed to detect any type of analyte, provided that it may be attached to the particles. However, it is preferred that the methods are performed using a fluid that comprises a sample containing the analyte. Typically, the sample comprises a crude lysate of solid tissue, a crude lysate of a cell of cells, or a body fluid. More preferably, the sample comprises blood or a blood product or component. Most preferably, the sample comprises whole blood or blood plasma. Generally, the sample is from a mammal, such as a human. The term “analyte” is not particularly limiting. Suitable analytes may be any type of biomolecule which it is desired to detect in a sample. For example, the analyte may be a protein, prion, peptide, carbohydrate, DNA or RNA, or whole cell, virus or bacteria. In particular, the analyte may be an antigen, a viral protein, a bacterial protein, an antibody, a specific DNA and/or RNA sequence, or specific cell type. In specific embodiments of the present invention the analyte is related to the diagnosis and treatment (including the determination of theranostic information) of Hepatitis C, HIV or other viral pathogens.

The term “sample” is not especially limiting and refers to any specimen in which an analyte may be present. In particular, as already mentioned, the sample may be whole blood, urine or other body fluid, or a crude lysate of solid tissue or cells or supernatant from cultured cells. The sample may be subjected to processing steps before it is used in the present method.

The recognition agents referred to in the methods of the present invention are not especially limited. The particles may be coated with the recognition agent. The nature of the recognition agent is not especially limited, provided that it allows the particle to bind specifically to a target analyte. The recognition agent may be an antibody, specific for an antigen which may itself be the target analyte, or may be an antigen present on the surface of the target analyte. Alternatively, where the target analyte is a polynucleotide the recognition agent may be a polynucleotide sequence complementary to a section of the sequence of the analyte. In a further embodiment the recognition agent may be a lectin where the analyte is a carbohydrate. In a further embodiment the recognition agent may be a cell surface receptor and the analyte its ligand, agonist or antagonist. In a further embodiment the recognition agent may be an antibody or ligand and the analyte a cell or living organism with the appropriate antigenic determinant or receptor. Where there are two or more analytes under investigation, an antibody specific for each analyte may be employed, to ensure that one particle type attaches to one analyte and a different particle type attaches to another analyte. In this manner, a plurality of analytes can be processed in the same sample.

In the present methods the particle that is buoyant in the fluid is not especially limited. Buoyant particles suitable for use in the present invention are also commercially available. In particular the buoyant particle may be a hollow glass bead.

Magnetic particles suitable for use in the present invention are well known in the art. In particular, magnetic beads are commercially available for magnetic separation in a variety of sizes. In one embodiment the beads are super-paramagnetic beads. Preferably the particles do not have any remnant magnetism when not placed in a magnetic field to prevent aggregation of the particles, and to ease dispersal and mixing within the fluid.

The particles may also comprise a label to aid with their detection. Preferably, the label is a fluorescent label.

The detection element for detecting an analyte may comprises any detection element, provided that the element is suitable for detecting the analyte under investigation. Preferably, the element comprises one or more of a biosensor array, an electrochemical biosensor element, and an optical biosensor element.

In a further preferred embodiment of this method, in one or more detecting conduits, an analyte may be concentrated according to a concentrating method as described above.

The invention also provides a method for detecting one or more analytes, which method comprises:
(a) separating an analyte, according to a method as defined above; and
(b) detecting the one or more analytes.

Further provided is a method of diagnosing the presence of a pathogen in a subject, or detecting the presence of a genotype in a subject, which method comprises:
(a) obtaining a sample from the subject;
(b) detecting the absence or the presence and/or quantity of the pathogen, or detecting the absence presence and/or quantity of a protein a polypeptide or a nucleic acid characteristic of the genotype, in the sample according to a method as defined above; and
(c) making a diagnosis of the subject, or determining the absence or presence of the genotype, based on the absence or the presence and/or quantity of the pathogen, or based on the absence or the presence and/or quantity of the polypeptide or nucleic acid characteristic of the genotype.

In this method, the pathogen is typically selected from a bacterium and a virus, or wherein the polypeptide is selected from a protein or a protein fragment, or the nucleic acid is selected from DNA and RNA. More preferably, the pathogen is HCV, HIV, rhinovirus, influenza virus or herpes virus. Typically, the subject is a mammal, such as a human. Typically the above listed viruses are human viruses.

Yet further provided is an apparatus for separating two or more analytes in a fluid, which apparatus comprises:
(a) a binding zone;
(b) two or more functional conduits;
(c) a separating conduit connecting the binding zone to the two or more functional conduits;
(d) a transporter for transporting the analyte through the separating conduit from the binding zone to the two or more functional conduits; and
(e) optionally one or more concentrating zones in connection with at least one of the functional conduits;
   wherein, in use, the functional conduits are situated each at differing heights, allowing buoyant particles to move from a lower conduit to a higher conduit as fluid flows through the separating conduit into the functional conduits.

The apparatus of the invention is typically a flow cell type apparatus. In the apparatus of the present invention, the transporter generally comprises a pump for pumping the fluid from the binding zone.

Preferably the apparatus comprises at least one detecting element in at least one of the functional conduits. It is further preferred that the one or more detecting elements are situated above one or more concentrating zones. Typically, the detecting element is a biosensor or a micro array.

The invention will now be described by way of example only, with reference to the following specific embodiments.

### EXAMPLES

Protocol for Samples that are to be Tested for HCV (this Protocol may also be Applied to HBV, HAV, HIV, Human Rhinovirus, Influenza Virus or Herpes Simplex Virus)

Nature of the Sample

Typically the sample is whole blood, serum, plasma, cell lysate or extraction (such as B cells or hepatocytes), nasopharyngeal mucous or urine. The sample may be conditioned to have a certain buffer composition, depending on the sample-type and its specific nature.

Bead Preparation

1 μg biotinylated antibody (other recognition agents, such as, oligonucleotides, PCR fragments, aptamers, PNA, lectins, antibody fragments, recombinant or purified receptors, and proteins may be employed as desired) to HCV El protein in 100 μl Phosphate Buffered Saline (PBS) or 1 μg biotinylated recombinant core protein antigen in 100 μl Phosphate Buffered Saline (PBS) or 1 μg biotinylated antibody to human Alanine aminotransferase (ALT) or 1 μg biotinylated antibody to human Aspartate aminotransferase (AST); are coupled to batches of 300 μl buoyant (MST technologies) beads of different densities at 20×10⁶ beads/ml that have been coated with streptavidin by the manufacturer. The high affinity of biotin for streptavidin (K_{d}=10⁻¹⁴M) ensures a successful reaction and allows the antibodies to coat the surface of the beads. The reaction is washed of excess uncoupled antibody by centrifuging the beads for 5 min at 14,000 rpm, discarding the supernatant and replacing with fresh PBS. This wash step is repeated twice.

Binding Step

The sample with a volume of 1-5 ml is incubated for several minutes with the beads that are coupled with antibodies that have been raised to HCV surface proteins, HCV core proteins or biomarkers of liver health such as AST and ALT. This can also be achieved online, by flowing the sample at a rate of 0.1 to 5 ml/min over the beads in a chamber that allows retention of the sample (fritting material or filter) but permits the flow of solutions (preferred method). Through the same channel wash solution is passed after the sample, in a volume of 3 to 5 times the volume of the sample, to eliminate non-specific binding. This wash solution may contain detergents such as Triton X-100, Tween 20 or Nonidet P40 at concentrations of 0.01 to 1% that reduce the non-specific binding that can be observed in antibody-antigen interactions.

Flow through to Sorting Mechanism or Detection Area

A valve on the microfluidic system is opened to allow the flow through of particles towards the sorting area. Using low flow rates (0.01 to 1 ml/min) the beads are flowed through the system towards the system comprising fluids of different densities. These fluids of differing densities could be easily obtained by adding density agents such as glycerol to the serum. In such a case, lower percentages of glycerol would be diluted into the higher conduits into a buffer such as PBS supplemented with 1% BSA and a detergent such as TritonX-100 or Nonidet P40. During the flow step, depending on the geometry of the channels and the buoyancy of the beads, the particles that have bound the relevant entities are sorted into the relevant channels for detection and/or separation. Briefly, when within a particular conduit a bead has a density inferior to the fluid in which it is surrounded, it will migrate to the superior surface of the conduit. When the conduit opens to communicate with the conduit situated directly above it, the bead can migrate into this conduit according to its lower density. Should the bead be neutrally buoyant in this system, it will remain in the conduit. The process takes place in a similar manner for all the conduits, until all the different batches of beads with differing densities are sorted, with their attached analytes. If the mechanism is purely used to separate the beads, they are taken to a collection chamber where further processing, if that is required, can take place. The density of the fluids and beads may be from 1 to 1.3 g/cm³.

If the beads are taken to a detection point or biosensor, they are flowed past it again at a low flow rate. The biosensor is equipped with antibodies raised against another antigenic epitope of the captured analyte. Once bound, the beads that have not bound any biosensor recognition sites are flushed away using a wash solution, similar to that mentioned above.

Detection

If the beads are fluorescent they can be detected and counted immediately using a microscope or CCD camera. If the beads are not fluorescent a secondary antibody, raised to the primary antibody used on the bead, tagged with fluorescent molecule or an enzyme capable of generating a chemiluminescent signal (such as horseradish peroxidase—HRP) can be used (impedence methods, or enzymatic electrochemical detection methods may also be employed). This is flowed over the bead complex at a concentration of approximately 0.5 μg/ml. It is important that the secondary antibody does not cross-react or recognise the biosensor recognising entity. Detection is achieved by measuring the fluorescence emitted by the reaction using a microscope or a CCD camera.

1. A method for separating two or more analytes in a fluid, which method comprises:
(a) binding each different analyte to a different particle in a binding zone, to produce two or more bound analytes;
(b) allowing the bound analytes to move through a separating conduit to two or more separate functional conduits;
wherein each different particle has, or can be controlled to have, a different buoyancy in the fluid as compared with the other particles; and wherein the separating conduit is in fluid communication with the two or more functional conduits, each functional conduit being situated at a different height from the other functional conduits; and each functional conduit containing a fluid having a different fluid density from the fluids in the other functional conduits such that each different particle when attached to an analyte has neutral buoyancy in at least one of the functional conduits, thereby allowing separation of the bound analytes by means of the neutral buoyancies of the different particles in the different functional conduits.

2. A method according to claim 1, wherein each different particle is attached to a different recognition agent that is specific for a different analyte.

3. A method according to claim 1, wherein the particles are selected from:
(a) particles that are buoyant in the fluid;
(b) magnetic particles whose buoyancy can be controlled by the application of a magnetic field;
(c) particles that are more dense than the fluid; and/or
(d) particles that are neutrally buoyant in the fluid.

4. A method according to claim 3, wherein in one or more detecting conduits, an analyte is concentrated by:
(a) allowing the bound analyte to rise towards one or more functional zones above the bound analyte by means of a particle to which the analyte is bound that is buoyant in the fluid, thereby concentrating the bound analyte in the vicinity of the one or more functional zones; and/or
(b) allowing the bound analyte to sink towards one or more functional zones below the bound analyte by means of a particle to which the analyte is bound that more dense than the fluid, thereby concentrating the bound analyte in the vicinity of the one or more functional zones.

5. A method according to claim 2, wherein one or more of the recognition agents comprise an antibody.

6. A method according to any preceding claim 1, wherein the particles that are buoyant in the fluid comprise one or more hollow glass beads.

7. A method according to claim 1, wherein the fluid comprises a sample containing the analyte.

8. A method according to claim 7, wherein the sample comprises a crude lysate of solid tissue, a crude lysate of cells, a body fluid, blood or a blood product.

9. A method according to claim 8, wherein the sample comprises whole blood or blood plasma.

10. A method according to claim 8, wherein the sample is from a mammal.

11. A method according to claim 10 wherein the sample is from a human.

12. A method according to claim 1, further comprising a detection element for detecting an analyte comprises one or more of a biosensor array, an electrochemical biosensor element, and an optical biosensor element.

13. A method according to claim 1, wherein the analyte is selected from a biological molecule, a virus or virus component, and a cell or a cell component.

14. A method according to claim 13, wherein the analyte comprises a protein, a polypeptide, a prion, a carbohydrate, a lipid, DNA and/or RNA.

15. A method for detecting one or more analytes, which method comprises:
(a) separating an analyte, according to a method as defined in any preceding claim; and
(b) detecting the one or more analytes.

16. A method of diagnosing the presence of a pathogen in a subject, or detecting the presence of a genotype in a subject, which method comprises:
(a) obtaining a sample from the subject;
(b) detecting the absence or the presence and/or quantity of the pathogen, or detecting the absence presence and/or quantity of a protein a polypeptide or a nucleic acid characteristic of the genotype, in the sample according to a method as defined in claim 15; and
(c) making a diagnosis of the subject, or determining the absence or presence of the genotype, based on the absence or the presence and/or quantity of the pathogen, or based on the absence or the presence and/or quantity of the polypeptide or nucleic acid characteristic of the genotype.

17. A method according to claim 16, wherein the pathogen is selected from a bacterium and a virus, or wherein the polypeptide is selected from a protein or a protein fragment, or the nucleic acid is selected from DNA and RNA.

18. A method according to claim 17, wherein the pathogen is an HCV, HIV, rhinovirus, influenza virus or herpes virus.

19. A method according to claim 16, wherein the subject is a mammal.

20. A method according to claim 19 wherein the subject is human.

21. An apparatus for separating two or more analytes in a fluid, which apparatus comprises:
(a) a binding zone;
(b) two or more functional conduits;
(c) a separating conduit connecting the binding zone to the two or more functional conduits;
(d) a transporter for transporting the analyte through the separating conduit from the binding zone to the two or more functional conduits; and
(e) optionally one or more concentrating zones in connection with at least one of the functional conduits;
wherein, in use the functional conduits are situated each at differing heights, allowing buoyant particles to move from a lower conduit to a higher conduit as fluid flows through the separating conduit into the functional conduits.

22. An apparatus according to claim 21, further comprising at least one detecting element in at least one of the functional conduits.

23. An apparatus according to claim 22, comprising one or more detecting elements above one or more concentrating zones.

24. An apparatus according to claim 21, wherein the transporter comprises a pump for pumping the fluid from the binding zone.

25. An apparatus according to claim 22, wherein the detecting element is a biosensor or a microarray.
